(19) United States
(12) Patent Application Publication
(10) Pub. No.: US 2007/0003626 A1
(43) Pub. Date: Jan. 4, 2007

(54) Sterile in-situ microcarrier forming gelled polymeric dispersions and processes to manufacture the same

(76) Inventor: Daniel Hendrix, Stuttgart (DE)

Correspondence Address: FOLEY AND LARDNER LLP;SUITE 500, 3000 K STREET NW, WASHINGTON, DC, 20007, US

(21) Appl. No.: 10/574,233

(22) PCT Filed: Jan. 6, 2004
(86) PCT No.: PCT/IB04/00003

§ 371 Date: Mar. 31, 2006

(51) Int. Cl. A61K 9/14; A61K 47/30

(52) U.S. Cl. 424/486; 514/772.3

(57) ABSTRACT

These inventions is in the field of drug delivery systems comprising of sterile gelling agents and in-situ polymeric gelled dispersions which maybe microparticulate for parentral administration and contain a therapeutically active component. They may release the active agent immediately or over a period of time.

### FIELD OF THE INVENTION

This invention is in the field of manufacture of sterile in-situ microparticulate forming gelled polymeric dispersions for parenteral administration. Also, described are gelled polymeric dispersions containing bioactive agents for their controlled or immediate release for the treatment of maladies.

### BACKGROUND OF THE INVENTION

Conventional liquid formulations have been used in the past for the parenteral administration of bioactive agents for the treatment of a variety of disease conditions in human beings and animals. Such formulations include simple aqueous or non-aqueous solutions or suspensions, lyophilized powders for reconstitution for administration via routes such as intravenous, intraarterial, subcutaneous, intramuscular and the like; solid implants for subdermal administration; microencapsulated products for intramuscular administration and the like. More recently, research has focused on the development of controlled release compositions which form the delivery systems inside the body after administration. Such compositions include the ATRIGEL system described by Dunn et al. (U.S. Pat. No. 4,938,763), the ReGel system developed by Rathi et al. (PCT Application WO 00/18821) and others. Even more recently, delivery compositions which form microcarriers inside the body have been developed to overcome the problems associated with the delivery systems described above. Such problems include prolonged processing times, use of costly equipments, use of toxic and often carcinogenic organic solvents and subsequent problems associated with their removal from the composition and the like. These and other problems are overcome by in-situ microcarrier forming delivery compositions as described by Bhagwatwar et al. (US 20030049320 A1, AU 0222505 A5 and WO 02/49573 A3).

All of these compositions are required to be sterile, endotoxin and foreign particulate free to reduce the probability of infections to the animal because of contamination by microorganisms. The term “Foreign particulate free” is meant to indicate the absence of any particulate matter, which is not supposed to be there in the composition and excludes drug particles, controlled release microparticulates and the like. Conventional methods for the manufacture of sterile compositions include sterilization by moist heat (autoclaving), sterilization by dry heat, ethylene oxide sterilization (gaseous sterilization), exposure to ultraviolet rays or to gamma irradiation or sterilization by aseptic processing. These and other methods of sterilization are described in detail in Pharmaceutical Dosage Forms : Parenteral Medications (Eds. Avis, Lachman and Lieberman, Volumes 1-3).

Most of the conventional formulations for parenteral administration described above can be readily processed by one of the manufacturing processes mentioned above and the choice of an appropriate method for their sterilization is within the scope of understanding of a person of ordinary skill in the art of manufacture of parenteral dosage forms. Thus, solution formulations of bioactive agents which are stable to temperature can be readily autoclaved post-processing of the formulation and filling into the final container. This process known as “terminal sterilization” is generally used for ensuring sterility of large volume parenterals such as normal saline, dextrose saline and the like. For temperature labile bioactive agents the formulations have to necessarily be processed aseptically that is through filtration through sterilizing grade filters which have a nominal pore size of 0.22 μm. Similarly, such agents which are sensitive to temperature and also to water can also be filled into vials, ampoules or syringes and then lyophilized. Lyophilized products which are free from moisture are then reconstituted before administration providing a prolonged shelf-life. Other compositions can be sterilized by ethylene oxide or by irradiation. Each of these methods suffers from disadvantages such as residual ethylene oxide, degradation due to heat or irradiation and others.

It is especially difficult to manufacture sterile controlled release products for parenteral administration such as the microencapsulated products, in-situ forming implants and the in-situ microcarrier forming gelled polymeric dispersions. Of all of the controlled release products mentioned above, sterile processing of the in-situ microcarrier forming gelled polymeric dispersions poses the greatest challenge to the formulation scientist because of the complex nature of the delivery composition. These gelled polymeric dispersions are comprised of dispersions of organic solvent solutions of biocompatible polymers in a continuous oleaginous phase gelled and stabilized by emulsifiers chosen from sorbitan monostearate or monopalmitate. The organic solvents are preferably water-soluble though water-immiscible solvents can also be used. Upon coming in contact with an aqueous medium, the oily continuous phase gets emulsified and the polymers from the droplets precipitate through the mixing and extraction of water-soluble solvents from the droplets. The detailed composition and processes to make the compositions are described in US 20030049320 A1, AU 0222505 A5 and WO 02/49573 A3 to Bhagwatwar et al. and are all incorporated herein by reference.

Though different methods to prepare sterile compositions are described in the literature, the literature is silent with respect to sterile in-situ microcarrier forming gelled polymeric dispersions and processes to manufacture such compositions. Problems associated with the sterile processing of the in-situ microcarrier forming gelled polymeric dispersion compositions include: instability of the polymer to heat, moisture and gamma irradiation, difficulty of aseptic processing of drug-free or drug-containing polymer solutions of high concentrations of greater than 40% w/w of polymer and up to 50% w/w of bioactive agent with respect to the polymer, and preparation of the sterile gelling agent bulk sorbitan monostearate or sorbitan monopalmitate.

The gelling agents of this invention as commercially available are not free from foreign particulate material and contain significant quantities of impurities which add color to the final product making it unacceptable for parenteral use. Further, the use of water for the processing of these gelling agents is not feasible because the gelling agents would degrade during autoclaving for example resulting in a loss of gelling capability. Also, presence of moisture in any of the materials, specially the gelling agent would result in the loss of physical stability of the gelled dispersions.

A process to manufacture sterile in-situ microcarrier forming gelled polymeric dispersion compositions would be a tremendous improvement in the current state-of-the-art in the development of commercial products using these compositions.

There is thus a need for preparing a sterile bulk sorbitan monostearate or sorbitan monopalmitate gelling agent substantially free from moisture and foreign particulate matter thereby improving its utility value as a pharmaceutical entity. The sterile gelling agent and its processes of manufacture are described in Bhagwatwar et al (PCT Application No. PCT/IB03/04509) and are incorporated herein by reference.

There is a further need to provide a process for the manufacture of sterile in-situ microcarrier forming gelled polymeric dispersion compositions containing bioactive agents for their immediate or controlled release.

There is also a need to provide such sterile in-situ microcarrier forming gelled polymeric dispersion compositions for use in the treatment of various disease conditions in human beings and animals.

### SUMMARY OF THE INVENTION

A novel method for the manufacture of sterile in-situ microcarrier forming gelled polymeric dispersion compositions for parenteral administration, is described. The method involves the use of aseptic processing alone or gamma-irradiation alone or a combination of aseptic processing and gamma irradiation to achieve a product of the desired attributes including sterility, freedom from foreign particulates, syringeability, particle formation upon coming in contact with aqueous media, potency of active, and physical stability.

The present inventors have surprisingly found that the manufacture of a sterile in-situ microcarrier forming gelled polymeric dispersion composition with the desired characteristics is dependant on the successful preparation of a sterile gelling agent bulk which is free from foreign particulate matter and substantially free from moisture, while retaining its capability to gel and physically stabilize the gelled polymeric dispersion composition.

The gelling agents of the invention include sorbitan monostearate or sorbitan monopalmitate which are known to be soluble in a variety of organic solvents (both volatile and non-volatile) at elevated temperatures followed by gelation of the solvents upon cooling. This principle has been used in the development of the in-situ microcarrier forming gelled polymeric dispersion compositions by Bhagwatwar et al. (US 20030049320 A1, AU 0222505 A5 and WO 02/49573 A3). The present inventors further have also astonishingly observed that certain volatile organic solvents such as ethanol, methanol and the like, not only act as good solvents for the gelling agents at elevated temperature but are readily removable through simple vacuum drying, with or without application of heat, without any change in the product characteristics. Further such solvents also ensure the sterility of the finished bulk. In addition, a majority of the solvent can be removed through the addition of a non-solvent such as for example water for injection. One other property of the use of such solvents includes that when excess solvent is added to the gelled bulk upon cooling, the sterile bulk gelling material precipitates out. Also, the residual solvent, if any remains after this rigorous processing, is non-toxic as the solvents such as ethanol are acceptable for parenteral administration.

In another embodiment of the invention, a sterile gelling agent is prepared by subjecting a foreign particulate free gelling agent as processed above to gamma irradiation.

In another aspect of the invention a sterile polymer-drug solution is prepared by gamma irradiation of such a solution.

In yet another aspect of the invention, a in-situ microcarrier forming gelled polymeric dispersion composition prepared using the gelling agents treated as above are subjected to gamma irradiation to prepare a sterile product useful for human administration without any significant loss in potency or behavior of the gelled dispersion composition in physical stability, syringeability or particle formation upon coming in contact with aqueous media.

Another embodiment of the invention describes the administration of this sterile composition to an animal species.

These and other embodiments of the invention are described in greater detail in the further sections of this application.

### DETAILED DESCRIPTION OF THE INVENTION

Sterile in-situ microcarrier forming gelled polymeric dispersion compositions for parenteral administration and methods for their manufacture, are provided. The process for the preparation of the sterile gel requires the preparation of a sterile, foreign particulate free, residual moisture free bulk gelling agent.

Preparation of a Sterile Gelling Agent

The gelling agents of the invention include sorbitan monostearate and/or sorbitan monopalmitate. Both of these materials are available from a number of suppliers commercially for topical or oral use. Sterile material free from foreign particulates is not available and is difficult to produce. It is now possible to produce such sterile, foreign particulate free material through the use of aseptic precipitation and drying alone or in conjunction with gamma irradiation. It is essential that the process used for the preparation of a sterile bulk does not introduce any change in physicochemical properties of the gelling agent which will in turn cause a change in the gelling potential of the gelling agent.

The process to provide a foreign particulate free, sterile bulk gelling agent comprises the steps of:
1. Dissolution of the gelling agent(s) in volatile organic solvents, if required at an elevated temperature, followed by filtration of the solutions also at the elevated temperature and subsequent evaporation to obtain a dry product OR
2. Dissolution of the gelling agent(s) in water-miscible organic solvents at an elevated temperature followed by filtration of the solutions also at the elevated temperature, precipitation of the gelling agents from solution either through addition of a non-solvent or cooling the solution to a lower temperature to cause phase-separation, filtration to recover the wet bulk and subsequent evaporation to obtain a dry product AND
3. Subjecting the foreign particulate-free gelling agent obtained from steps 1 and 2 to sterilization by gamma irradiation

The gelling agents of the invention viz. sorbitan monostearate or sorbitan monopalmitate are soluble in a variety of organic solvents including volatile organic solvents such as methylene chloride, ethyl acetate, benzene, petroleum ether, carbon tetrachloride, methanol, acetonitrile, acetone, ethanol, tetrahydrofuran and other volatile solvents, at elevated temperatures. Non-volatile organic solvents in which the gelling agents are soluble include NN′-dimethylacetamide (DMA), dimethylsulfoxide (DMSO), N-methyl-2-pyrrolidone (NMP), triacetin, triethyl citrate, benzyl alcohol, propylene carbonate, decylmethylsulfoxide, dimethylformamide (DMF), glycofural, benzoyl benzoate, alkyl esters of aromatic acids, polyethylene glycols (PEG), propylene glycol and the like, also at elevated temperatures.

The solvents for the invention should be biocompatible and not pose toxicity issues. Such solvents may be completely water-miscible, partially water-miscible or completely water-immiscible. Water-immiscible organic solvents such as dichloromethane, chloroform, ether, benzene, hexane and the like though otherwise used in the preparation of pharmaceutical compositions are generally toxic and require exotic methods such as lyophilization and the like for removal. Also, there are very strict guidelines for the levels of such residual solvents allowable in pharmaceutical compositions, especially so for parenteral administration. Water-miscible organic solvents are preferred. More specifically, such water-miscible organic solvents are chosen from DMA, DMSO, NMP, triacetin, triethyl citrate, benzyl alcohol, propylene carbonate, decylmethylsulfoxide, DMF, glycofural, benzoyl benzoate, alkyl esters of aromatic acids, PEG, propylene glycol, isopropanol, methanol, acetonitrile, acetone, ethanol, tetrahydrofuran and the like. A specially preferred volatile water-miscible solvent is ethanol because of its proven antiseptic properties, its complete water-miscibility, its volatile nature, its biocompatibility and low toxicity potential, its solvating capability for the gelling agents of the invention at elevated temperatures and its known use in the pharmaceutical industry for parenteral administration of solution dosage forms. Preferred non-volatile water-miscible organic solvents include DMA, DMSO, DMF, NMP, and PEG among others.

In one embodiment of the novel process of the invention, the sterile bulk gelling agent is prepared by dissolution in volatile water-miscible organic solvents specifically chosen from ethanol, methanol, acetonitrile, acetone, ethanol, tetrahydrofuran at a temperature above ambient, filtered through a sterilizing grade filter membrane at the elevated temperature and dried under vacuum at the elevated temperature.

In another aspect of this embodiment of the invention the solution of the gelling agent which has been filtered through a sterilizing grade filter membrane, is cooled to room temperature or lower to cause gelation of the solvent and the same solvent is added to the gel to cause precipitation of the gelling agent. This suspension is then filtered through a further sterilizing grade filter membrane and the wet mass which is substantially free from the organic solvent is then dried under vacuum at an elevated temperature. This procedure has the advantage of removal of a large percentage of the organic solvent from the gelled bulk which would otherwise be lost under vacuum. This solvent can then further be used in processing of a separate lot of the gelling agent. The term “substantially free” indicates that a large percentage of the organic solvent is removed when compared with the situation where all of the solvent is bound to the gelling agent in the form of a gel.

In a further embodiment of the invention, the solution of the gelling agent which has been filtered through sterilizing grade filter membranes at elevated temperatures is then added to a non-solvent to cause precipitation of the gelling agent. The non-solvent can be an organic solvent or can be water. The precipitated gelling agent is then treated further as in the earlier embodiment described above with the same advantages.

In a further aspect of this embodiment, the water-miscible organic solvent is non-volatile chosen from DMA, DMSO, NMP, triacetin, triethyl citrate, benzyl alcohol, decylmethylsulfoxide, DMF, glycofural, PEG, propylene glycol and the like. Upon precipitation from a non-solvent such as water, the gelling agent is subjected to drying to remove water. The residual organic solvent should be chosen advantageously to be the same as the one to be used in the final gelled polymeric dispersion.

In an additional embodiment of the invention, the sterile dried bulk gelling agent is subjected to sterilization by gamma irradiation. Surprisingly, the gelling agents are not degraded by this procedure. Other materials in the gelled polymeric dispersion can be processed by other techniques as described above and generally known in the art of preparation of sterile dosage forms.

The concentration of the gelling agent in the water-miscible solvent can be from 5% w/w to 80% w/w, preferably from about 10% w/w to 70% w/w and even more preferably from about 20% w/w to 60% w/w. The concentration of the gelling agent in the final solution will be dependent on the solvent chosen, the gelling agent solvent interactions, the processing temperature and of course the solubility of the gelling agent in the solvent. Of particular importance is the concentration of the gelling agent in the solvent, which allows filtration through a sterilizing grade membrane filter. The higher the concentration of the gelling agent in the solution the greater the yields and smaller the amounts of the expensive solvents that are used.

When a solvent/non-solvent type of precipitation is to be used, the ratio of the solvent to the non-solvent may be adjusted so that complete precipitation occurs to ensure maximum yields.

The temperature of preparation of the solution and also the processing will of course depend on the solvent to be used with a temperature below the boiling point of the solvent being preferred. Thus, for example, for a solvent such as ethanol a clear solution can be prepared at a temperature of 35-40° C. and higher at concentrations as high as 50% w/w, which can be filtered through a sterilizing grade 0.22 μm filter at the same temperature. But, for a solvent such as DMA, a solution with the same concentration needs to be processed at a higher temperature because of the viscosity imparted by the gelling agent.

The gelling agents of the invention are known to gel the solvents of the invention at high concentrations. Each gelling agent-solvent system will have a different temperature range at which the gelation occurs which will also affect the processability of the solution.

The sterilizing grade filter can be any membrane, which has the capability to remove foreign particulates and also microorganisms to ensure sterility. Such filters usually have a pore size of 0.22 μm. Any membrane filter is acceptable for the practice of this invention as long as it can filter the solution and is compatible with the solvents of the invention. Such membranes include those made from nylon 66, cellulose acetate, cellulose nitrate, polytetrafluoroethylene (PTFE), silver membrane, gold membrane, polysulfone, polycarbonate and other known in the art and supplied by various manufacturers. The sterilizing grade filter could be preceded by a cleaning filter such as a 0.45 μm, 5 μm or 8 μm filter which can take up much of the burden from the sterilization filter. The choice of filters is within the scope of a person skilled in the art of development of pharmaceutical injectable dosage forms.

Any mode of filtration is acceptable as long as a sterile product is produced. Such methods include, vacuum filtration, filtration under positive pressure using compressed air or nitrogen and the like. Also, the use of cartridge filters or filter candles and the like are well within the scope of this description.

The drying of the sterile gelling agent bulk obtained after the above described processes can be conducted by any means known in the art including for example tray drying in an oven with or without the application of vacuum and with or without heating, lyophilization, simple vacuum drying and other methods known to a person skilled in the art of processing pharmaceutical dosage forms. Where heating is required it is preferable to heat at a temperature above the boiling point of the solvent to be evaporated to ensure complete removal of the organic solvent.

Further, for the preparation of a sterile in-situ microcarrier forming gelled polymeric dispersion composition, the preparation of sterile solvents, polymers, oil and bioactive agents can be as per known procedures in the art such as aseptic filtration of the solvents and oil, gamma irradiation of the polymer or aseptic filtration of the polymer solution in a volatile organic solvent followed by evaporation of the solvent and the like and are well known to persons skilled in the art of manufacture of parenteral controlled release dosage forms.

In a specific embodiment of this invention, a solution of the drug and the polymer in the solvents of the invention can be subjected to gamma irradiation to achieve a sterile drug-polymer solution useful for further processing. Similarly, the bioactive agent may be subjected to gamma irradiation sterilization and then used as a sterile bulk for further processing.

Polymers, solvents, oils, emulsifiers and other agents which are useful in the context of this particular composition and its sterile product are described in US 20030049320 A1, AU 0222505 A5 and WO 02/49573 A3 to Bhagwatwar et al. and are all incorporated herein by reference.

The bioactive agents which can be incorporated into the sterile in-situ microcarrier forming gelled polymeric dispersion compositions can be chosen from peptide drugs, protein drugs, desensitizing agents, antigens, vaccines, anti-infectives, antibiotics, antimicrobials, antineoplastics, antitumor, antiallergenics, steroidal anti-inflammatory agents, analgesics, decongestants, miotics, anticholinergics, sympathomimetics, sedatives, hypnotics, antipsychotics, psychic energizers, tranquilizers, androgenic steroids, estrogens, progestational agents, humoral agents, prostaglandins, analgesics, antispasmodics, antimalarials, antihistamines, cardioactive agents, non-steroidal anti-inflammatory agents, antiparkinsonian agents, antihypertensive agents, beta-adrenergic blocking agents, nutritional agents, antivirals, DNA fragments, nucleic acids, genetic material, oligonucleotides, radioisotopes, or combinations of these classes of compounds. To those skilled in the art, other drugs or biologically active agents that can be released in an aqueous environment can be utilized in the described delivery system. Also, various forms of the drugs or biologically active agents may be used. These include, without limitation, forms such as uncharged molecules, molecular complexes, salts, ethers, esters, amides, and other chemically modified forms of the biologically active agent which are biologically activated when injected into a body.

The gelled polymeric dispersion can then be finally compounded aseptically by the processing steps comprising:
1. Providing a solution of the sterile biocompatible polymer in the sterile biocompatible organic solvent at an elevated temperature, with or without a bioactive agent;
2. Providing a sterile continuous oil phase by dissolving the sterile gelling agent prepared by one of the techniques described above in the oil phase at the same temperature as the polymer solution phase
3. Combining the polymer solution of (1) above with the continuous oil phase of (2) above with homogenization to form a hot dispersion
4. Gradually cooling the hot dispersion prepared in (3) above to refrigeration temperature to form the sterile gelled dispersion
5. Filling the sterile in-situ microcarrier forming gelled polymeric dispersion composition as prepared by steps 1-4 into prefilled syringes or vials or other packaging material as desired and
6. Optionally, subjecting the sterile packaged product prepared by steps 1-5 to terminal gamma irradiation sterilization.

In an additional embodiment of the invention, the gelled dispersion filled into the final packaging material is subjected to gamma irradiation to achieve terminal sterilization. The source of irradiation and the dose of gamma irradiation which is essential for assuring complete sterility of the composition, the polymers, polymer solutions, the polymer-drug solutions or the dry bioactive agents is within the scope of understanding of a person skilled in the art of irradiation sterilization. Further, a dose of 25 KGy is accepted internationally as a sufficient dose to ensure sterility.

In a further embodiment, the sterile bioactive agent may also be added to the oil phase as a suspension to enhance the loading of the bioactive agent in the delivery composition. Whatever the final composition that is arrived at the process allows a sterile composition to be prepared. Also, the further behavior of the delivery composition in forming a delivery system and the subsequent release of the bioactive agent is as described in US 20030049320 A1, AU 0222505 A5 and WO 02/49573 A3 to Bhagwatwar et al. and are all incorporated herein by reference.

The term “parenterally” as used herein is intended to include routes such as intramuscular, intravenous, subcutaneous, subdermal, intralesional, intratumoral, intracavitary, peritumoral, intraarticular, vaginal, intraperitoneal, intraabdominal, intrathecal, intraorgan and the like or on open wounds, fractures, ulcers, cancerous lesions and the like and is not to be construed as limiting on the scope of the invention. Thus, the composition can be used for the immediate or controlled release or both of bioactive agents or bioinactive agents wherever the use of a sterile composition is called for.

Thus, the sterile gelled dispersion prepared as described above can be filled into prefilled syringes either preattached with needles or a separate sterile needle may be provided along with to be attached before administration parenterally.

For rectal or vaginal administration or for colonic delivery through the rectum the sterile gelled dispersion could be formulated into a suppository or a pessary. For such formulation the sterile gelled dispersion could be poured into refrigerated suppository molds of predetermined sizes. Upon cooling, the suppositories could be removed and packaged into laminated aluminum pouches to be stored under refrigerated conditions. Of course, to enhance the consistency of the composition to form a suppository, the concentration of the sterile gelling agent in the composition could be increased as desired. The determination of the exact percentage composition of the sterile gelling agent required in the sterile gelled dispersion for the formation of a suppository or pessary is well within the scope of a pharmaceutical formulation scientist versed in the art of development of pharmaceutical dosage forms.

For the use of the sterile gelled dispersion composition in the treatment of burns or open wounds such as fractures or during treatment of open lesions, the sterile gelled dispersion may be filled into sterile collapsible tubes or presterilized wide mouth containers.

The formulation of the sterile gelled composition as described above and its further use in the treatment of different maladies is intended to include any other application wherever the sterile composition could be used and should not be construed as limiting on the scope of the description provided herein.

Further, the sterile gelling agents prepared as per the processes described herein could be used in the preparation of other sterile compositions such as creams, ointments, pastes, cosmetics for specialized applications, niosomes, liposomes, proliposomes, in-situ forming non-polymeric delivery systems, other sterile compositions where the sterile gelling agents may find use as emulsifiers such as emulsions, solid lipid nanoparticles and the like without any limitation.

The following examples describe the invention in further detail and are not to be construed as limiting on the scope of the invention.

### EXAMPLES

### Comparative Example 1

### Preparation of a Placebo Gelled Polymeric Dispersion Using Non-Sterile Gelling Agent

A placebo gelled dispersion was prepared as follows. A poly-DL-lactide-co-glycolide polymer (Comonomer ratio 75:25) was dissolved in a solvent phase comprising DMA:PEG 400 (25:75% w/w) by heating at 80° C. on an oil bath to make a 40% w/w polymer solution. The temperature of polymer phase was reduced to 65° C. This solution was then emulsified into an oil phase comprising sorbitan monostearate (Sanyo, Japan), 6.0 g and polysorbate 80 (Croda), 0.4 g, in 13.6 g sesame seed oil (Croda) also held at 65° C., aided by homogenization at 16,000 rpm speed using a Ultra-Turrax T-25-basic homogenizer for 3 minutes. The speed of homogenization was reduced to 11,000 rpm and the hot dispersion was cooled to 2-8° C. with the aid of continued homogenization.

This gelled dispersion was easily syringeable through an 18-gauge needle and readily (within 5-7 minutes) formed particles upon coming in contact with the aqueous medium. The dispersion was stable at 2-8° C.

The dispersion was subjected to a sterility test as per the method described in the United States Pharmacopoeia and failed in this test.

### Example 1

### Preparation of a Placebo Gelled Polymeric Dispersion Using Sterile Sorbitan Monostearate

A placebo gelled dispersion using sterile sorbitan monostearate was prepared as per the procedure described in Comparative Example 1.

This gelled dispersion also was easily syringeable through an 18-gauge needle and readily (within 5-7 minutes) formed particles upon coming in contact with the aqueous medium. The dispersion was stable at 2-8° C.

The behavior and characteristics of the gelled dispersion system prepared from the sterile sorbitan monostearate are comparable with the characteristics and behavior of the gelled dispersion system as per Comparative Example 1. The sterilization procedure for the gelling agent does not affect the characteristics of the gelling agent.

The gelled polymeric dispersion of Example 1 was subjected to sterility testing as per the procedure described in the United States Pharmacopoeia. The product passed this test for sterility.

### Example 2

### Preparation of a Sterile Gelled Polymeric Dispersion Containing Paclitaxel Using Sterile Sorbitan Monostearate

A paclitaxel containing gelled polymeric dispersion using sterile sorbitan monostearate was prepared as follows. A poly-DL-lactide-co-glycolide polymer (Comonomer ratio 75:25) was dissolved in a solvent phase comprising of DMA:PEG 400 (25:75% w/w) by heating at 80° C. on an oil bath to make a 40% w/w polymer solution. Paclitaxel, 10% w/w with respect to the polymer was added to the polymer solution held at 80-85° C. and mixed till dissolved. The rest of the processing was as per Comparative Example 1.

This gelled dispersion was easily syringeable through an 18 gauge needle and readily (within 5-7 minutes) formed particles upon coming in contact with the aqueous medium. The gelled dispersion contained 95.66±1.42% paclitaxel of the label claim (8 mg per gram of the gelled polymeric dispersion). The dispersion was stable at 2-8° C.

The use of sterile gelling agent did not affect the characteristics of a drug loaded gelled polymeric dispersion.

The gelled polymeric dispersion of was subjected to sterility testing as per the procedure described in the United States Pharmacopoeia. The product passed this test for sterility.

### Example 3

### Sterilization of Gelled Polymeric Dispersion Containing Paclitaxel by Gamma Irradiation

A paclitaxel containing gelled dispersion was prepared as per the earlier examples and subjected to gamma irradiation at a dose of 25 KGy.

The gelled dispersions before and after gamma irradiation sterilization were easily syringeable through an 18-gauge needle and readily (within 5-7 minutes) formed particles upon coming in contact with the aqueous medium. The gelled dispersion was analyzed for paclitaxel content by a HPLC method and was shown to contain 98.02±0.36% and 94.96±1.08% paclitaxel of the label claim (8 mg/g of gelled dispersion), before and after irradiation. The dispersion was stable at 25° C. for at least 7 days and for more than 2 months at 2-8° C.

These examples demonstrate that sterilization of the gelled polymeric dispersion had no impact on the characteristics of the dispersion. Additionally, the gelled dispersion thus prepared is sterile as a dose of 25 KGy is accepted internationally to ensure sterility.

### Example 4

### Sterilization of Paclitaxel Containing Solutions by Gamma Irradiation

Paclitaxel either as a powder or in solution in different solvents such as DMA, PEG 400, DMA:PEG 400 (25:75% w/w) and in a polymer solution in DMA:PEG 400 (25:75% w/w) was subjected to gamma irradiation sterilization at a dose of 25 KGy and analyzed for paclitaxel content before and after irradiation.

TABLE 1
Effect of gamma irradiation on paclitaxel
stability in various compositions

| | | Assay, % | | |
|---|---|---|---|---|
| | | | | WRT |
| | | | Post- | initial, |
| No. | Description | Initial | irradiation | % * |
| 1. | Paclitaxel | 100 | 94.74 ± 2.36 | 94.74 |
| 2. | Paclitaxel in DMA | 97.67 ± 1.21 | 92.60 ± 1.75 | 94.81 |
| 3. | Paclitaxel in PEG-400 | 97.53 ± 0.23 | 90.60 ± 0.35 | 92.89 |
| 4. | Paclitaxel in DMA:PEG- | 95.79 ± 1.11 | 90.41 ± 2.39 | 94.38 |
| | 400 (25:75, % w/w) | | | |
| 5. | Paclitaxel in the | 98.61 ± 1.24 | 99.38 ± 1.09 | 100.78 |
| | polymer solution | | | |

* WRT initial indicates the percentage remaining after treatment

The data indicate a marginal degradation of the paclitaxel in the solvents. The paclitaxel in the polymer solution demonstrated an assay of 100%.

### Example 5

### Administration of the Sterile Paclitaxel Containing Gelled Polymeric Dispersion to Nude Mice

The sterile gelled polymeric dispersion of Example 8 was administered intratumorally into nude mice xenografted with NT8 human head and neck cancer tumors. The animals did not display any signs of toxicity or infection even after 28 days.

Other inventors may be able through simple experimentation, to define different variants of the processes described in this application. All of these variants are meant to be encompassed within the scope of this invention without exception.

1. A pharmaceutical drug delivery system comprising of a sterile gelling agent, sterile excipients and additionally therapeutically active component, wherein the percentage of the sterile gelling agent in the final composition is sufficient to increase the viscosity of the medium.

2. The pharmaceutical drug delivery composition of claim 1 wherein the sterile gelling agent is chosen from fatty acids, esters of fatty acids, salts of fatty acids and fatty alcohols or mixtures thereof.

3. The sterile gelling agent of claim 2 which is preferably chosen from sorbitan monostearate, sorbitan monopalmitate, aluminum monostearate and cetostearyl alcohol or mixtures thereof.

4. The pharmaceutical drug delivery system of claim 1 wherein the gelling agent present is from about 0% -75% w/w, preferably from 5% to 50% w/w of the total composition, wherein the gelling agent may consist of a mixture of gelling agents.

5. The pharmaceutical drug delivery system of claim 1, which can consist of bioactive agents chosen from peptide drugs, protein drugs, desensitizing agents, antigens, vaccines, anti-infectives, antibiotics, antimicrobials, antineoplastics, antitumor, antiallergenics, steroidal anti-inflammatory agents, analgesics, decongestants, miotics, antyicholinergics, sympathomimetics, sedatives, hypnotics, antipsychotics, psychic energisers, tranquilizers, androgenic steroids, estrogens, progestational agents, humoral agents, prostaglandins, analgesics, antispasmodics, antimalarials, antihistaminics, cardioactive agents, non-steroidal anti-inflammatory agents antiparkinsonism agents, antihypertensive agents, beta-adrenergic blocking agents, nutritional agents, antivirals, DNA fragments, nucleic acids, genetic material, oligonucleotides, radioisotopes or combinations of these classes of compounds.

6. A pharmaceutical drug delivery system of claim 1, which is in the form of an in-situ microcarrier forming composition, an in-situ forming implant, a gel, a gelled dispersion, a cream, an ointment, a paste, a pessary, a suppository or a wound dressing.

7. The pharmaceutical drug delivery system according to claim 1, which can be administered by any route such as parenteral, oral, vaginal, rectal, nasal, intraocular, topical and onto open wounds.

8. The pharmaceutical drug delivery system according to claim 1, which can be administered by any parenteral route including intravenously, intramuscularly, subcutaneously, intratumorally, intracavitary peritumorally, intrathecally, subdermally, intrafat, intraabdominally, intralesionally, intracranially, onto open wounds, into fractures and into surgical sites.

9. A process of preparing a drug delivery system comprising of:
(a) Providing a solution of the sterile biocompatible polymer in a sterile biocompatible organic solvent, with or without a bioactive agent.
(b) Providing a sterile continuous oil phase by dissolving the sterile gelling agent in the oil phase.
(c) Combining (a) and (b) above to form a dispersion.
Cooling the dispersion obtained in (c) to form a sterile gelled dispersion and
Optionally, subjecting the drug delivery system prepared as above to terminal sterilization procedure preferably gamma irradiation.

10. A method for administering a pharmaceutically active moiety to a subject in need of such administration by using a drug delivery system of claim 1.

11. A method for administering a bioactive agent using a delivery system of claim 1, to a subject in need of such administration which comprises in
(a) providing a parenteral composition containing a sterile in-situ gelling agent in a sterile composition and
(b) assistance by the sterile gelling agent by processes of diffusion, degradation which may be independent or overlapping for the release of the therapeutically active moiety from the delivery system.
